(19) United States
(12) Patent Application Publication
(10) Pub. No.: US 2005/0059634 A1
(43) Pub. Date: Mar. 17, 2005

(54) Per-6-substituted-per-6-deoxy-cyclodextrins, and use of the same to inhibit soluble beta-amyloid-peptide derived oligomers and to treat alzheimer's and related diseases

(76) Inventors: Duane L. Venton, Lombard, IL (US); William L. Klein, Winnetka, IL (US); Gregory R.J. Thatcher, Chicago, IL (US); Lei Chang, Evanston, IL (US); Rong Liu, Evanston, IL (US); Zhiqiang Wang, Chicago, IL (US); Mark Holterman, River Forest, IL (US)

Correspondence Address: MARSHALL, GERSTEIN & BORUN LLP, 6300 SEARS TOWER, 233 S. WACKER DRIVE, CHICAGO, IL, 60606, US

(21) Appl. No.: 10/899,468

(22) Filed: Jul. 26, 2004

Related U.S. Application Data
(60) Provisional application No. 60/490,461, filed on Jul. 28, 2003.

(51) Int. Cl. A61K 31/724

(52) U.S. Cl. 514/58

(57) ABSTRACT

Per-6-substituted-per-6-deoxy-cyclodextrins and compositions containing the same are disclosed. The compounds and compositions inhibit the formation and/or activity of soluble β-amyloid-peptide derived oligomers, and can be used to treat diseases and conditions wherein such inhibition is beneficial, for example, Alzheimer's disease.

### CROSS REFERENCE TO RELATED APPLICATIONS

This application claims the benefit of U.S. Provisional Patent Application Ser. No. 60/490,461, filed Jul. 28, 2003.

### FIELD OF THE INVENTION

The present invention relates to per-6-substituted-per-6-deoxy-cyclodextrins (i.e., per-6-substituted-CDs herein), and compositions containing the same. The present invention also relates to a method of inhibiting the formation and/or activity of soluble β-amyloid-peptide (Aβ) derived oligomers in a mammal by administering a therapeutically effective amount of a per-6-substituted-CD of the present invention to the mammal. The present per-6-substituted-CD compounds, compositions containing the same, and methods are useful in the treatment of a variety of diseases and conditions, particularly Alzheimer's disease.

### BACKGROUND OF THE INVENTION

The fastest growing segment of the U.S. population is individuals aged 65 years and older. As a result of this demographic shift, the number of individuals aged 75 years is expected to triple, and the number of individuals over 85 years to double, over the next 30 years. Aging is associated with a progressive deterioration of the normal functions of an individual, in particular a decline in the function of the central nervous system (CNS), which results in impaired or hampered motor activities, and a compromised quality of life.

Aging also is an important risk factor for Alzheimer's disease (AD) and a variety of other degenerative diseases of the brain. AD is characterized by a progressive deterioration in cognitive performance, and is a fatal progressive dementia for which there is no cure and limited treatment. AD is the most common form of dementia in older individuals, affecting 5% to 10% of the population over the age of 65. One of the most under-addressed health problems today is an adequate method of preventing and treating AD.

A prominent feature of AD is the presence of extracellular neuritic plaques, which have lengthy fibrils constructed from Aβ monomers at their core. Therefore, increasing concentrations of Aβ can contribute to AD pathology. It has been proposed that neurodegeneration in AD is caused by deposition of Aβ in the plaques found in the brain tissue (A. Lorenzo et al., Proceedings of the National Academy of Science, USA, 91, 12243-12247 (1994); D. H. Small, “The amyloid cascade hypothesis debate: emerging consensus on the role of Aβ and amyloid in Alzheimer's disease,” The Sixth International Conference on Alzheimer's disease: Amsterdam, The Netherlands, 1998; pp 301-304 (1998)). However, a frequent objection to this hypothesis is that the number of amyloid deposits in the brain does not correlate well with the degree of cognitive impairment in transgenic mice or humans (R. D. Terry, “The neuropathology of Alzheimer disease and the structural basis of its cognitive alterations, in Alzheimer disease,” Lippincott Williams & Wilkins: Philadelphia, Pa., 187-206 (1999); L. Mucke et al., Journal of Neuroscience, 20, 4050-4058 (2000); W. L. Klein, “Fibrils, protofibrils & Aβ-derived diffusible ligands: how Aβ causes neuron dysfunction and death in Alzheimer's disease,” Hummana Press: Totowa, N.J., 1-49 (2001)).

Recent research based on transgenic models of AD has cast doubt on both the fibril dependence and irreversibility of memory loss. In one model, despite accelerated formation of detergent-insoluble aggregates of amyloid β peptide (Aβ) and early onset of memory decline, no correspondence could be shown between memory and Aβᵢₙₛₒₗ (M. A. Westerman et al., J. Neurosci., 22, 1858-1867 (2002)). More remarkably, recovery of memory function recently was reported for transgenic mice vaccinated with antibodies against Aβ (J. C. Dodart et al., Nat. Neurosci., 5, 452-457 (2002)). Such recovery, which occurred within a day of injection and without impact on insoluble amyloid fibrils, had been predicted by an alternative hypothesis for the structure and pathogenic role of Aβ-derived toxins (M. P. Lambert et al., Proc. Natl. Acad. Sci. U.S.A., 96, 3228-3233 (1999); W. L. Klein et al., Trends Neurosci., 24, 219-224 (2001)).

Recent studies also indicate that the most important role of Aβ in the progression of AD may not be beta-amyloid plaque formation, but the formation of intermediate, soluble oligomers (J. Hardy et al., Science (USA), 297, 353-356 (2002)). These soluble oligomeric proteins form in the brain of an individual suffering from AD and are variously termed “amyloid-beta-derived diffusible ligands,” “Alzheimer's disease diffusible ligands,” or “ADDLs.” Soluble, metastable Aβ₁₋₄₂ intermediates (i.e., ADDLs (Lambert, 1998) or protofibrils (D. M. Hartley et al., J. Neurosci. 19, 8876-8884 (1999)) cause subtle injury to cultured neurons. Microinjection of culture medium containing naturally secreted human Aβ into living rats revealed that the oligomers in the absence of monomer and amyloid fibrils can inhibit long-term potentiation in the hippocampus. Further, this effect was attributed specifically to the soluble oligomers of Aβ (D. M. Walsh et al., Nature, 416, 535-539, (2002)).

In this alternative hypothesis, a basis for reversible, fibril-independent memory loss lies in the neurological properties of soluble Aβ assemblies. Distinct from fibrillar amyloid, ADDLs (Lambert, 1998) and the somewhat larger, rod-shaped protofibrils (J. D. Harper et al., Annu. Rev. Biochem., 66, 385-407 (1997); D. M. Walsh et al., J. Biol. Chem., 272, 22364-22372 (1997)) are potent CNS neurotoxins (Hartley, 1999). The oligomers are especially relevant to memory dysfunction because they rapidly and selectively inhibit long-term potentiation (Lambert, 1998; Walsh, 2002; H. W. Wang et al., Brain Res., 924, 133-140 (2002)), an established paradigm for synaptic information storage.

Based on their impact on CNS models, it is clear that soluble Aβ assemblies can be an important factor in AD, putatively accounting for the discrepancies between dementia and amyloid plaque burden. Inhibition of the assembly or activity of ADDLs therefore is a strategy for a potentially effective prevention or treatment of AD. In particular, research indicates that the discovery of a drug that targets the assembly or activity of ADDLs represents an important approach to treating AD and related diseases and conditions.

For example, investigators recently have shown that Ginkgo biloba extracts inhibit β-amyloid-induced cell death by inhibiting the formation of ADDLs (Z. Yao et al., Brain Research, 889, 181-190 (2001)). However, little else is known concerning compounds that might act in this fashion.

One study shows that Aβ interacts with beta-cyclodextrin (hereafter “beta-CD”), which substantially diminishes the neurotoxic effects of Aβ₁₋₄₀ in PC12 cells (P. Camilleri et al., Federation of European Biochemical Societies Letters, 341, 256-258 (1994)). In addition, other studies demonstrate the protective effects of beta-CD in vivo (J. Waite et al., Neurobiology of Aging, 13, 595-599 (1992)). The effect of beta-CD on the ability to inhibit ADDLs formation was studied, but little effect on the formation of the soluble forms of this neurotoxin was found. Nevertheless, specific interactions of per-6-substituted-beta-CD libraries with other molecules was shown (J. Yu et al., Bioorganic Medicinal Chemistry Letters, 9, 2705-2710 (1999); J. Yu et al., “Combinatorial search for enzyme-like activity,” Abstr. Pap.-Am. Chem. Soc., 220th, MEDI-228 (2000)).

In particular, evidence suggests that AD may be caused by inflammatory processes associated with aging, and not, as generally believed, by plaque-like deposits in the brain. Amyloid plaques are hard, waxy deposits containing proteins and polysaccharides that result from the degeneration of tissue. For nearly two decades, Alzheimer's disease research has focused on compounds and methods to prevent the formation-of fibrils, which coalesce into even larger deposits in the brain, i.e., plaques, that many investigators believe kill nerve cells in the brain.

Presently, investigators still hypothesize that derivatives of the monomeric Aβ peptide is the root cause of AD, but many investigators have concluded that AD is attributed to the formation of toxic, soluble protein aggregates, rather than the buildup of plaque and tangles inside nerve cells in the brain. ADDLs have chemical and toxicological properties quite different from either single Aβ molecules or aggregations of these molecules, i.e., fibrils.

Also, unlike fibrils, ADDLs are highly selective in toxicity. ADDLs selectively, but not absolutely, affect particular types of brain cells that atrophy in AD patients. Fibrils, however, kill a broad range of nerve cells, including destroying cell types that remain healthy even until patients die. ADDLs also are soluble, which means ADDLs can diffuse throughout the brain. In contrast, fibrils are confined to the specific locations where they first form, and these locations correspond poorly with the brain areas that wither as AD progresses. It also has been suggested that ADDLs begin to interfere with the basic mechanism of long-term memory well before ADDLs attain levels sufficiently high to kill brain cells.

Representative publications directed to ADDLs and the relationship between ADDLs and AD include J. Yu et al., J. Mol. Neurosci, 19, 51-5 (2002); Wang, 2002; W. L. Klein, Neurochem Int, 41(5); 231-5 (2002); Yao., 2001; and Lambert, 1998. Also see, J. Hardy et al., “The amyloid hypothesis of Alzheimer's disease: progress and problems on the road to therapeutics,” Science (USA), 297, pages 353-356 (2002); G. A. Krafft et al., in Ann. Rep. Med. Chem., 37, Doherty, Ed., 31-40, Academic Press, San Diego, Calif. (2002); and U.S. Pat. No. 5,834,446, incorporated herein by reference.

Currently, no therapy is available for the prevention, treatment, or reversal of AD. Present-day therapies are hypothesized as slowing the progression of AD by increasing the efficacy of the remaining neurons in the brain, and these therapies appear to perform best in the early stages of AD. Current AD drugs relieve symptoms, but do not treat the underlying pathology. Moreover, they improve cognition and maintain patient function only for a limited time.

The four AD therapies presently approved for mild to moderate AD, i.e., ARICEPT, EXELON, REMINYL, and COGNEX, have estimated sales in 2002 of $1.1B. These four drugs are all acetylcholinesterase inhibitors (AChEIs), the first drugs specifically indicated for AD, but only at the mild to moderate level of severity. Current therapy still lacks efficacy because 36% of patients fail first-line therapy and 44% of patients fail second-line therapy.

Another proposed therapy, an antibody vaccine, was beneficial to two-thirds of the patients in a clinical trial. This result shows that patients who generate antibodies exhibit significantly slower rates of decline of cognitive functions and daily activities. Therefore, antibodies against Aβ plaques can slow cognitive decline in patients with Alzheimer's disease. However, the antibody vaccine failed the clinical trial because the drug caused brain inflammation and death.

In view of the foregoing, it would be a significant advance in the art to provide compounds, compositions, and methods that inhibit the formation and/or activity of ADDLs, and, consequently, are useful in the treatment of AD and other diseases and conditions associated with ADDLs. The present invention discloses per-6-substituted-CDs that inhibit the formation of ADDLs, and are useful in the treatment and prevention of AD and related diseases and conditions.

### SUMMARY OF THE INVENTION

The present invention is directed to the administration of a per-6-substituted-CD of the present invention to an individual in need thereof to treat Alzheimer's disease and related diseases. In particular, the present invention is directed to per-6-substituted-CDs that inhibit the formation and/or activity of ADDLs, and to compositions containing the same.

The present invention also is directed to a therapeutic use of the compounds and compositions containing the same by administration of an ADDL inhibitor to an individual in need thereof to treat a condition or disease wherein inhibition of the ADDLs formation or activity provides a benefit, for example, in the treatment or prevention of AD or pre-AD disorders, such as mild cognitive impairment.

Accordingly, one aspect of the present invention is to provide per-6-substituted-CDs and compositions containing one or more per-6-substituted-CDs. A present compound or composition provides a method of treating or preventing AD when administered in a therapeutically effective amount to an individual in need thereof. The per-6-substituted-CDs bind to Aβ, and have been shown to inhibit the formation of neurotoxic aggregants. The present per-6-substituted-CDs, contrary to a previously tested vaccine, are not expected to induce transient encephalitis.

In one embodiment of the invention, the per-6-substituted-CD comprises a per-6-substituted-beta-CD. In a further embodiment, the per-6-substituted-CD comprises a per-6-substituted-alpha-CD.

In another aspect of the present invention, alterations in brain amyloid activity are modulated by passage of the active agent across the blood brain barrier. The present benzyl and furfurylamine beta-CDs, i.e., compounds 4b and 4a, can be improved by modification to increase transport across the blood brain barrier. As discussed in detail below, compounds 4b and 4a can be derivatized in a fashion that retains the anti-ADDL activity, while being capable of transport across the blood brain barrier.

In another embodiment, per-6-substituted-CDs that do not appreciably penetrate into the brain can provide clearance of neurotoxic aggregates from the brain by providing a peripheral link. It has been shown that antibodies against Aβ, induced by active immunization with Aβ peptides, reduce brain Aβ burden in amyloid-forming mice. Although enhanced microglial phagocytosis via Fc receptors might represent one plausible explanation, it has been suggested that antibodies present in the peripheral blood may alter the central nervous system/peripheral Aβ equilibrium. For example, the natural product, gelsolin, which is known to bind Aβ, but that is unrelated to an antibody or immune modulator, reduced brain levels of Aβ. As such, agents designed to bind with specificity and affinity to Aβ₁₋₄₂, but that are not capable of crossing the blood brain barrier still represent useful AD agents.

Modifying the primary hydroxyl face of the CD molecule may maximize affinity and specificity for the Aβ₁₋₄₂ molecule. Based on the above considerations, these agents may be active in their own right. Alternatively, modification of the secondary hydroxyl face of the molecule may provide agents that not only recognize the Aβ₁₋₄₂ molecule with affinity and specificity, but that cross the blood brain barrier.

Hydrophilic CDs are poorly absorbed, but CDs substituted with hydrophilic residues on the secondary hydroxyl face are readily absorbed from the gastrointestinal tract. CDs covalently attached to opioid receptor peptides, with methyl groups on the remaining hydroxyl groups to increase lipophilicity, were reported to cross the blood brain barrier.

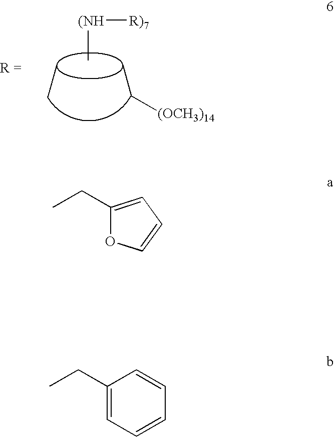

### Beta-CD Derivatives for Passage through the Blood Brain Barrier

The corresponding O-methyl-derivative of furfurylamine beta-CD 4a, i.e., derivative 6a in unpurified form (as well as the corresponding benzylamine derivative 6b) were prepared. Preliminary biological assays indicate that the methyl derivative 6a is at least as active as the free hydroxy derivative 4a, and perhaps as much as ten times more active in inhibiting ADDLs formation. These compounds are considerably more hydrophobic than the parent-free hydroxyl form, being fully soluble in methylene chloride and other organic solvents.

Still another aspect of the present invention is to provide a method of treating neurodegenerative diseases and conditions attributed to ADDLs and related soluble peptide aggregates. The method comprises administering a therapeutically effective amount of a per-6-substituted-CD to an individual in need thereof. The neurodegenerative diseases include, for example, Alzheimer's disease, Parkinson's disease, Huntington's disease, Creutzfeldt-Jacob disease, and spinocerebellar ataxias.

Another aspect of the present invention is to provide an article of manufacture for human pharmaceutical use, comprising (a) a package insert providing instructions for the treatment of AD, (b) a container, and (c) a packaged composition comprising a per-6-substituted-CD of the present invention, alone or with a second therapeutically active agent useful in a treatment for AD.

These and other aspects and novel features and advantages of the present invention will become apparent from the following detailed description of the preferred embodiments.

### BRIEF DESCRIPTION OF THE DRAWINGS

FIG. 1 illustrates the structure of beta-cyclodextrin (beta-CD);

FIG. 2 illustrates the synthesis of beta-CDs;

FIG. 3 is a reversed phase HPLC chromatogram of reaction products from a reaction between per-6-iodo-6-deoxy-beta-CD and furfurylamine;

FIG. 4 contains dot-blot and Western-blot ADDLs inhibition assays for beta-CD reaction products; and

FIG. 5 contains dose response plots of % inhibition of ADDLs formation (a) and % increase of ADDLs formation (b) vs. log ([CD]/[A-beta]).

### DETAILED DESCRIPTION OF THE PREFERRED EMBODIMENTS

Recent studies have shown that the most important role of Aβ in the etiology of AD may not be plaque formation, but in the formation of soluble, metastable Aβ₁₋₄₂ neurotoxic oligomers (i.e., ADDLs). Inhibiting the assembly or activity of ADDLs therefore represents an attractive target for the treatment of AD and related diseases and conditions. The present invention discloses the preparation, isolation, and evaluation of per-6-substituted-CDs that inhibit ADDL formation, and, accordingly ADDL activity.

The per-6-substituted-CDs of the present invention have a structure schematically illustrated below as (2a) and (2b), and are prepared by reacting the per-iodo-beta-CD (1) with a primary or a secondary amine. 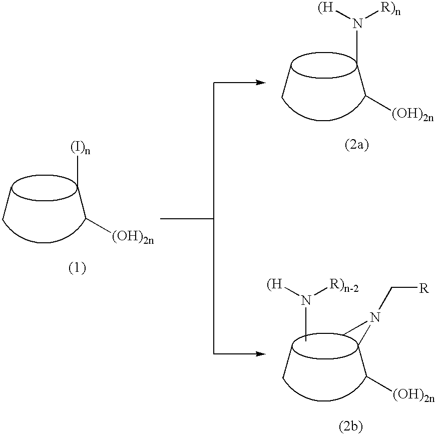
wherein n is 6 or 7 (2b)

The structure 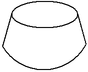
is an abbreviated structure for a cyclodextrin (CD) framework. The full structure of a beta-CD is shown, for example, in. U.S. Pat. No. 5,834,446, incorporated herein by reference.

In accordance with the present invention, the R groups are derived from an aromatic amine. The R groups of structures (2a) and (2b) can be, for example, 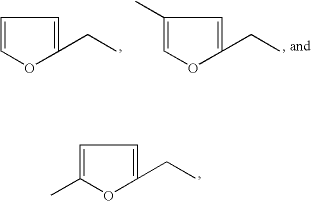
derived from furfurylamine, 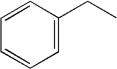
derived from benzylamine, or 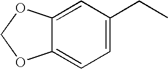
derived from 3,4-dioxymethylene substituted benzyl-amine. The benzylamine substituted per-6-substituted beta-CD is disclosed in J. Org. Chem., 62(25), 8760-8766 (1997). Typically, the R group is —CH₂-aryl or —CH₂-heteroaryl, wherein the aryl or heteroaryl group optionally is substituted. For example, the aryl or heteroaryl group is 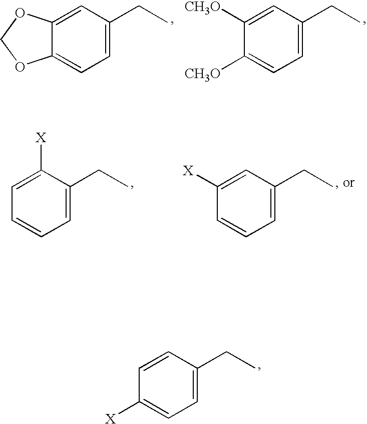
wherein X is selected from the group consisting of Cl, Br, CH₃, C₂H₅, and OCH₃.

In another embodiment, the per-6-substituted-beta-CDs have a structure (3a). 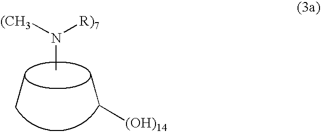
wherein the R group is defined above as in structures (2a) and (2b).

The present invention also is directed to the administration of a per-6-substituted-CD of the present invention, or a salt or prodrug thereof, to inhibit the formation and/or activity of ADDLs, and to treat or prevent AD and other diseases or conditions attributed to ADDLs. Related diseases include neurodegenerative conditions and dementia associated with aggregation of peptides in rain regions, with formation of aggregates that show broad similarity to that observed in AD. Examples include, but are not limited to, Huntington's Disease (HD) and Parkinson's Disease (PD), in which disease progression is associated with peptide aggregates, e.g., deriving from Huntington protein or forming Lewy bodies. Several characteristics of aggregation, including propensity for formation of beta-sheet structures, are shared with amyloid aggregation. Additional conditions treatable by the present invention include Creutzfeldt-Jacob disease, spinocerebellar ataxias, and similar neurodegenerative diseases.

The present invention also provides pharmaceutical compositions comprising a per-6-substituted-CD of the present invention. Further provided are articles of manufacture comprising a per-6-substituted-CD of the present invention, and an insert having instructions for using the compound.

The methods described herein benefit from the use of a present per-6-substituted-CD in the treatment and prevention of AD. A per-6-substituted-CD can be administered alone, or together with a second therapeutic agent useful in the treatment of Alzheimer's disease, to achieve a desired effect.

For the purposes of the invention disclosed herein, the term “treatment” includes preventing, ameliorating, or eliminating AD. As such, the term “treatment” includes both medical therapeutic and/or prophylactic administration, as appropriate.

The term “container” means any receptacle and closure therefor suitable for storing, shipping, dispensing, and/or handling a pharmaceutical product.

The term “insert” means information accompanying a product that provides a description of how to administer the product, along with the safety and efficacy data required to allow the physician, pharmacist, and patient to make an informed decision regarding use of the product. The package insert generally is regarded as the “label” for a pharmaceutical product.

The term “prodrug” means compounds that transform rapidly in vivo to a compound useful in the invention, for example, by hydrolysis. A thorough discussion is provided in Higuchi et al., Prodrugs as Novel Delivery Systems, Vol. 14, of the A.C.S.D. Symposium Series, and in Roche (ed.), Bioreversible Carriers in Drug Design, American Pharmaceutical Association and Pergamon Press, 1987.

The per-6-substituted-CDs of the present invention, i.e., the active agent, can be formulated in suitable excipients for oral administration, or for parenteral administration. Such excipients are well known in the art. The active agent typically is present in such a composition in an amount of about 0.1% to about 75% by weight, either alone or in combination.

Pharmaceutical compositions containing the active agents, i.e., the per-6-substituted-CDs of the present invention, are suitable for administration to humans or other mammals. Typically, the pharmaceutical compositions are sterile, and contain no toxic, carcinogenic, or mutagenic compounds which cause an adverse reaction when administered.

The method of the present invention can be accomplished using an active agent as described above, i.e., a per-6-substituted-CD of the present invention, or as a physiologically acceptable salt, derivative, prodrug, or solvate thereof. The active agent, or a form thereof, including a prodrug, can be administered as the neat compound, or as a pharmaceutical composition containing either or both entities. Administration of the pharmaceutical composition can be performed before or after the onset of AD or a related disease or condition.

The active agent can be administered by any suitable route, for example by oral, buccal, inhalation, sublingual, rectal, vaginal, intracisternal through lumbar puncture, transurethral, nasal, percutaneous, i.e., transdermal, or parenteral (including intravenous, intramuscular, subcutaneous, cutaneous, and intracoronary) administration. Parenteral administration can be accomplished using a needle and syringe, or using a high pressure technique, like POWDERJECT™.

The pharmaceutical compositions include those wherein the active ingredient is administered in an effective amount to achieve its intended purpose. More specifically, a “therapeutically effective amount” means an amount effective to prevent development of, or to abate or eliminate, AD. Determination of a therapeutically effective amount is well within the capability of those skilled in the art, especially in light of the detailed disclosure provided herein.

A “therapeutically effective dose” refers to that amount of active agent that results in achieving the desired effect. Toxicity and therapeutic efficacy of an active agent can be determined by standard pharmaceutical procedures in cell cultures or experimental animals, e.g., determining the LD₅₀ (the dose lethal to 50% of the population) and the ED₅₀ (the dose therapeutically effective in 50% of the population). The dose ratio between toxic and therapeutic effects is the therapeutic index, which is expressed as the ratio between LD₅₀ and ED₅₀. A high therapeutic index is preferred. The data obtained from such data can be used in formulating a range of dosage for use in humans. The dosage of the active agent preferably lies within a range of circulating concentrations that include the ED₅₀ with little or no toxicity. The dosage can vary within this range depending upon the dosage form employed, and the route of administration utilized.

The exact formulation, route of administration, and dosage is determined by an individual physician in view of the patient's condition. Dosage amount and interval can be adjusted individually to provide levels of the active agent that are sufficient to maintain therapeutic or prophylactic effects.

The amount of pharmaceutical composition administered is dependent on the subject being treated, on the subject's weight, the severity of the affliction, the manner of administration, and the judgment of the prescribing physician.

Specifically, for administration to a human in the curative or prophylactic treatment of AD, oral dosages of an active agent generally is about 2 to about 800 mg daily for an average adult patient (70 kg), typically divided into two to three doses per day. Thus, for a typical adult patient, individual tablets or capsules contain about 0.1 to about 500 mg active agent, in a suitable pharmaceutically acceptable vehicle or carrier, for administration in single or multiple doses, once or several times per day. Dosages for intravenous, buccal, or sublingual administration typically are about 0.1 to about 10 mg/kg per single dose as required. In practice, the physician determines the actual dosing regimen that is most suitable for an individual patient, and the dosage varies with the age, weight, and response of the particular patient. The above dosages are exemplary of the average case, but there can be individual instances in which higher or lower dosages are merited, and such are within the scope of this invention.

The active agents of the present invention can be administered alone, or in admixture with a pharmaceutical carrier selected with regard to the intended route of administration and standard pharmaceutical practice. Pharmaceutical compositions for use in accordance with the present invention thus can be formulated in a conventional manner using one or more physiologically acceptable carriers comprising excipients and auxiliaries that facilitate processing of the active agents into preparations which can be used pharmaceutically.

These pharmaceutical compositions can be manufactured in a conventional manner, e.g., by conventional mixing, dissolving, granulating, dragee-making, emulsifying, encapsulating, entrapping, or lyophilizing processes. Proper formulation is dependent upon the route of administration chosen. When a therapeutically effective amount of the active agent is administered orally, the composition typically is in the form of a tablet, capsule, powder, solution, or elixir. When administered in tablet form, the composition can additionally contain a solid carrier, such as-a gelatin or an adjuvant. The tablet, capsule, and powder contain-about 5% to about 95% of an active agent of the present invention, and preferably from about 25% to about 90% compound of the present invention. When administered in liquid form, a liquid carrier, such as water, petroleum, or oils of animal or plant origin, can be added. The liquid form of the composition can further contain physiological saline solution, dextrose or other saccharide solutions, or glycols. When administered in liquid form, the composition contains about 0.5% to about 90% by weight of active agent, and preferably about 1% to about 50% of an active agent.

When a therapeutically effective amount of the active agent is administered by intravenous, cutaneous, or subcutaneous injection, the composition is in the form of a pyrogen-free, parenterally acceptable aqueous solution. The preparation of such parenterally acceptable solutions, having due regard to pH, isotonicity, stability, and the like, is within the skill in the art. A preferred composition for intravenous, cutaneous, or subcutaneous injection typically contains, in addition to a compound of the present invention, an isotonic vehicle.

Suitable active agents can be readily combined with pharmaceutically acceptable carriers well-known in the art. Such carriers enable the active agent to be formulated as tablets, pills, dragees, capsules, liquids, gels, syrups, slurries, suspensions and the like, for oral ingestion by a patient to be treated. Pharmaceutical preparations for oral use can be obtained by adding the active agents with a solid excipient, optionally grinding the resulting mixture, and processing the mixture of granules, after adding suitable auxiliaries, if desired, to obtain tablets or dragee cores. Suitable excipients include, for example, fillers and cellulose preparations. If desired, disintegrating agents can be added.

The active agents can be formulated for parenteral administration by injection, e.g., by bolus injection or continuous infusion. Formulations for injection can be presented in unit dosage form, e.g., in ampules or in multidose containers, with an added preservative. The compositions can take such forms as suspensions, solutions, or emulsions in oily or aqueous vehicles, and can contain formulatory agents such as suspending, stabilizing, and/or dispersing agents.

Pharmaceutical compositions for parenteral administration include aqueous solutions of the active agent in water-soluble form. Additionally, suspensions of the active agent can be prepared as appropriate oil-based injection suspensions. Suitable lipophilic solvents or vehicles include fatty oils or synthetic fatty acid esters. Aqueous injection suspensions can contain substances which increase the viscosity of the suspension. Optionally, the suspension also can contain suitable stabilizers or agents that increase the solubility of the compounds and allow for the preparation of highly concentrated solutions. Alternatively, a present composition can be in powder form for constitution with a suitable vehicle, e.g., sterile pyrogen-free water, before use.

The active agent also can be formulated in rectal compositions, such as suppositories or retention enemas, e.g., containing conventional suppository bases. In addition to the formulations described previously, the compounds also can be formulated as a depot preparation. Such long-acting formulations can be administered by implantation (for example, subcutaneously or intramuscularly) or by intramuscular injection. Thus, for example, the active agents can be formulated with suitable polymeric or hydrophobic materials (for example, as an emulsion in an acceptable oil) or ion exchange resins, or as sparingly soluble derivatives, for example, as a sparingly soluble salt.

In particular, the active agent can be administered orally, buccally, or sublingually in the form of tablets containing excipients, such as starch or lactose, or in capsules or ovules, either alone or in admixture with excipients, or in the form of elixirs or suspensions containing flavoring or coloring agents. Such liquid preparations can be prepared with pharmaceutically acceptable additives, such as suspending agents. An active agent also can be injected parenterally, for example, intravenously, intramuscularly, subcutaneously, intrathecally, intracisternally, or intracoronarily. For parenteral administration, the active agent is best used in the form of a sterile aqueous solution which can contain other substances, for example, salts, or monosaccharides, such as mannitol or glucose, to make the solution isotonic with blood.

For veterinary use, the active agent is administered as a suitably acceptable formulation in accordance with normal veterinary practice. The veterinarian can readily determine the dosing regimen and route of administration that is most appropriate for a particular animal.

### Experimentals

Using a high throughput dot blot assay recently developed for measuring Aβ oligomerization (L. Chang et al., J. Neuromol. Chem., 20, 305-313 (2003)), various beta-CD derivatives were evaluated for an ability to inhibit oligomerization of Aβ₁₋₄₂. beta-CD (1) is a cyclic glucopyranose oligomer (FIG. 1), whose shape is reminiscent of a lamp-shade, frequently depicted schematically as compound 2 in FIG. 1. Actually three different hydroxyls are present in each sugar in the beta-CD molecule, which can be readily differentiated chemically. Additionally, no plane of symmetry exists in the molecule because the cyclic α-1,4-linkage forms a torus. Consequently, the primary hydroxyl groups are not equivalent when substituted. The end result of these structural features is that symmetry operations are not possible with different substitution patterns and each pattern represents a different compound. This asymmetry frequently provides closely related compounds that are difficult to separate, and also greatly complicates spectral interpretation in all but the per-substituted cases.

Chemistry Beta-CD derivatives were prepared by treatment of per-6-iodo-per-6-deoxy-beta-CD (1) with the appropriate amine under conditions described herein. Per-6-bromo-per-6-deoxy-beta-CD compounds also can be used in the present invention. Preparative reversed phase HPLC chromatography provided the homogeneous per-substituted derivatives (a) as well as crosslinked derivatives (b), with observed singly charged ESMS ions as indicated. In several cases wherein the bromo CD was used, a homogeneous per-6-substituted-CD was obtained without the need for chromatography.

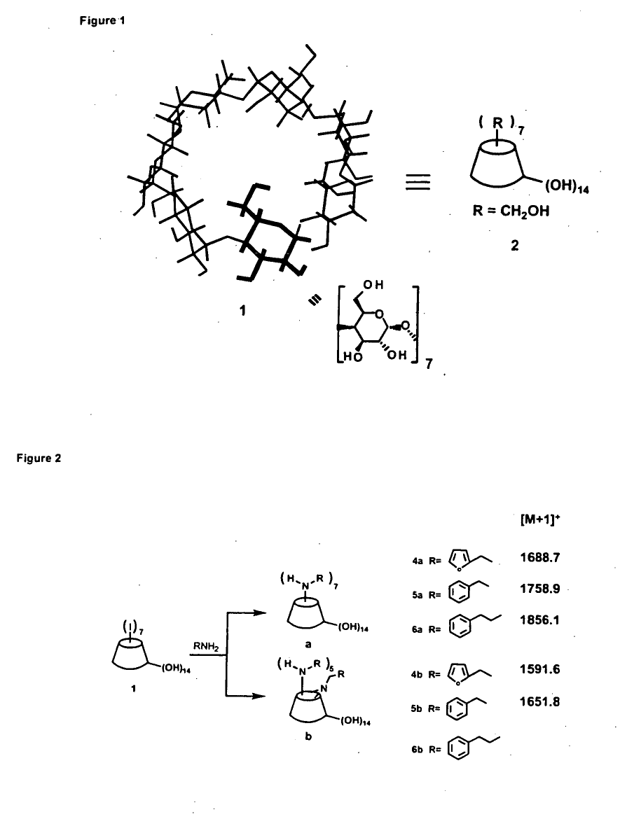

Per-6-substituted-beta-CDs of the present invention were prepared under nitrogen by treating a solution of per-6-iodo-beta-cyclodextrin (1 of FIG. 2, 300 mg, 0.157 mmol) with a primary or secondary amine, e.g., benzylamine or furfurylamine (3 mL), at room temperature for 120 hours, then heating at 80° C. to 85° C. for 6 hours (FIG. 2). After the removal of excess amine under reduced pressure at room temperature, the residue was solidified with ethyl acetate. The solidified residue was filtered, ultrasonicated in ethyl acetate, then filtered and dried to provide the crude products as off-colored powders. Analytical HPLC (C₁₈ column 3.9×15 mm, 4 μM; solvent: (A) water/0.1% TFA (trifluoroacetic acid), (B) CH₃CN (acetonitrile)/0.1% TFA, linear gradients (A to 92% B over 40 minutes, then to 95% B over 5 minutes) with a constant 3% MeOH (methanol) at 0.5 ml/min (milliliter/minute) gave two major peaks for both the furfurylamine (18.6, 19.7 min) and benzylamine (21.6, 23.1 min) beta-CD reaction products.

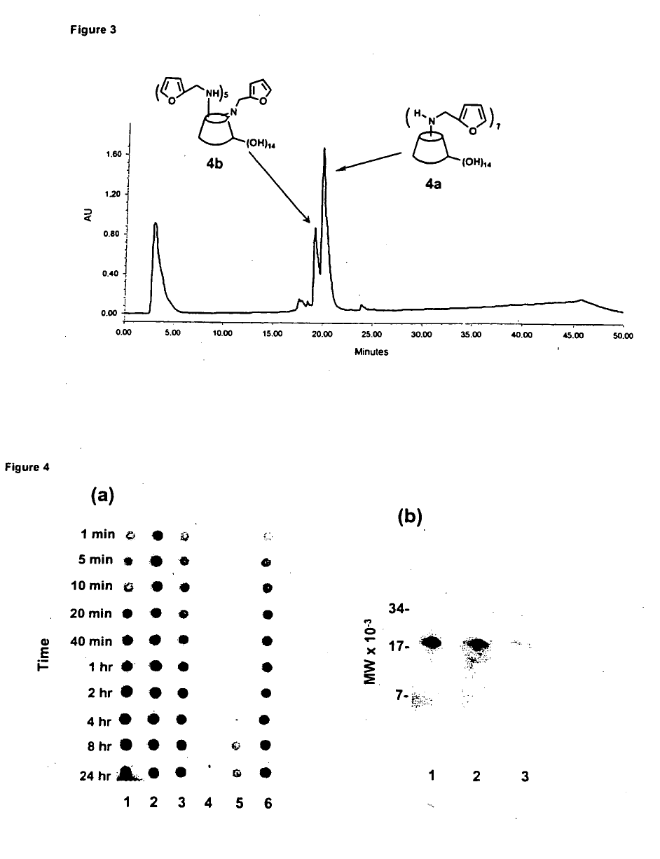

FIG. 3 illustrates a representative example of an HPLC chromatogram of the products produced in these reactions, in particular for the furfurylamine derivatives. Preparative reversed phase HPLC (C₁₈ column 15×300 mm, 100 Å, about 20 mg loading) provided near baseline separation of the products in both cases, as shown by analytical reversed-phase chromatography. Each of the four products were subjected to ESMS and ¹³C and ¹H NMR analyses. The NMR data was fully consistent with the proposed structures for the per-substituted-per-6-deoxy-beta-CDs 4a and 5a. In addition, the ESMS of these derivatives showed [M+H]⁺ ions (along with doubly charged ions) consistent with the proposed structures (see FIG. 2). The mass of the second HPLC peak in both cases suggested that after six nucleophilic displacements of the iodine, an internal displacement occurred to give the crosslinked products 4b and 5b. However, the NMR data for the proposed crosslinked products 4b and 5b could not be fully interpreted because of the multiplicity of signals caused by the asymmetry in the molecule. The compounds can be prepared free of the crosslinked product (e.g., 4b) by using per-6-deoxy-per-6-bromo-beta-CD as the starting material. By use of the same procedures for preparation and purification, the per-6-substituted phenethyl derivative 6a (26.1 min retention time under the same analytical HPLC conditions described above) also was prepared and tested.

ADDLs assay The assays were performed as set forth in Wang et al., J. Med. Chem., 47(13), pages 3329-3333 (2003). In short, an aliquot of Aβ₁₋₄₂ was dissolved in anhydrous DMSO (dimethyl sulfoxide) to a concentration of 22.5 μg/ml (5 mM), pipette mixed, and further diluted into ice-cold F12 medium (phenol red free) (Biosource CA) to make a 0.5 μM stock solution. The mixture was vortexed quickly, incubated at 6° C. to 8° C. for 24 hours, centrifuged at 14,000×g for ten minutes, then the oligomers were collected from the supernatant. Time-dependent ADDLs formation was monitored by diluting 4 μl of 5 mM or 0.5 mM Aβ/DMSO solutions with 196 μl of ice-cold F12 medium to 100 nM and 10 nM Aβ, respectively. These Aβ solutions were incubated at 4° C., then, at the indicated timepoint, 2 μl applied to nitrocellulose for analysis by dot-blot. Beta-CD derivatives were dissolved in ice cold F12 media at the specified concentrations. These F12/CD solutions then were used in ADDLs assays, and the oligomer formation monitored as described. Also see J. Yu et al., M. Mol. Neurosci., 19, 51-55 (2002) for a description of the ADDLs assay protocol.

FIG. 4(a) contains Dot-blot assays performed to measure ADDL formation over a period of 24 hours. The assays were performed on unpurified reaction products containing mainly persubstituted beta-CDs (a) and their corresponding crosslinked derivatives (b). Lane: 1, control; 2, imidazole reaction products at 20 μM; 3; N,N-dimethylethylenediamine reaction products at 20 μM ; 4-6; furfurylamine reaction products at 20, 2 and 0.2 μM, respectively. FIG. 4(b) is a Western blot of lane 4 at 4 hour time point.

ADDLs Western blot/dot blot assays For Western blots, samples were subjected to SDS-PAGE on 16.5% Tris-tricine gels at 100V (volumes) for 1.5 to 2 hours. Proteins then were transferred to nitrocellulose cellulose at 100V for 1 hour in the cold. For dot blots, nitrocellulose was prewetted with 20 mM Tris-HCl, pH 7.6, 137 mM NaCl (TBS) and partially dried. Samples then were applied to nitrocellulose and air dried completely. The nitrocellulose membranes then were blocked in 0.1% TWEEN 20 in TBS (TBS-T) with 5% nonfat dry milk powder for 1 hour at room temperature. The samples were incubated for 1 hour at room temperature with primary antibody M93-3 in the blocking buffer (1:1000), and washed 3×15 min with TBS-T. Incubation with HRP-conjugated secondary antibody (1:50,000) in TBS-T for 1 hour at room temperature was followed by washing. Visualization of proteins with chemiluminescent reagents was recorded by exposure to ECL film (Amersham-Pharmacia).

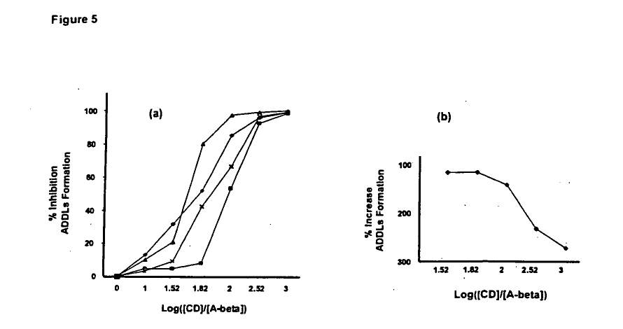

Dose response curves Dose response curves were prepared by performing the aforementioned dot-blot assays with Aβ₁₋₄₂ (10 nM) in the presence of purified per-6-substituted-beta-CDs at the indicated concentrations. The response was determined for the dots at 4 hours using Kodak 1D imaging software and reported as log dose response of the ratio of beta-CD to Aβ₁₋₄₂ as shown in FIG. 5.

FIG. 5 shows the dose-dependent inhibition of ADDL formation with purified beta-CD derivatives. FIG. 5(a) shows inhibition of ADDL formation by densitometric measurement of dot-blot assays at 4 hours for various concentrations: of purified beta-CD derivatives: ^{▴}, persubstituted furfurylamine 4a; ▪, crosslinked furfurylamine 4b; x, crosslinked benzylamine derivative 5a; and ♦, per-substituted benzylamine derivative 5b. FIG. 5(b) shows an increase in ADDL formation with reaction products from per-6-iodo-6-deoxy-beta-CD and phenethylamine 6(a) and (b).

In a previous publication (Yu, 2002), the preparation and testing of libraries of per-6-substituted-beta-CD derivatives for an ability to inhibit ADDL formation was disclosed. The libraries were prepared from per-6-iodo-per-6-deoxy-beta-CD by the simultaneous displacement of iodine with amine neucleophiles used three at a time (P. R. Ashton, Journal of Organic Chemistry, 61, 903-908 (1996)). In examining several of these libraries (each containing about 2000 derivatives) there was an indication that the inhibitory activity was a function of the type of amine used for preparation of the particular library. The most active library tested was derived from imidazole, N,N-dimethylethylenediamine, and furfurylamine, which at 20 μm total library (based on the anticipated average molecular weight of the derivatives), inhibited ADDL formation (10 nm, Aβ₁₋₄₂) over a period of four hours.

Because these libraries were complex mixtures of beta-CD isomers (as well as side products from the reaction), the present investigation was initiated by assaying the beta-CD products from the displacement with amines used individually as nucleophiles (see FIG. 4a for representative results). These assays were performed using the previously mentioned dot-blot assay (Yu et al., 2002).

It was found that products from the reaction with furfurylamine had significant activity (FIG. 4a, lane 4-6), while that from the imidazole (FIG. 4a, lane 2) and N,N-dimethylethylenediamine (FIG. 4a, lane 3) demonstrated almost no activity.

As shown in the Western blot assay (FIG. 4b), this activity appears to largely inhibit the tetrameric form of the ADDLs (18,056 Daltons). This led the present investigators to study the displacement products (analyzed by ESMS) from a variety of individual side chains in reaction with the iodo beta-CD for inhibition of ADDLs formation. Reaction products with all aliphatic amines tested showed no detectable activity. On the other hand, aromatic side chain reactants showed a highly variable activity. Thus, whereas the benzylamine products showed significant inhibition, even to 24 hours at 2 μM (based on the molecular weight of the per-substituted product), beta-CD products from the reaction with pyridine were essentially inactive. Further, reaction products with phenethylamine had diagrammatically the opposite effect, i.e., resulting in stimulation of ADDLs formation (see following discussion and FIG. 5b). The same type of activity was found in the furfurylamine series, with one of the more active derivatives in this series being furfurylamine itself. However, placing a methyl group on the furan ring led to reduced activity, while a methyl group on the nitrogen gave products with as good or better activity than the furfurylamine beta-CD. Finally, saturation of the furfuryl amine ring dramatically reduced the ability of these derivatives to inhibit ADDLs formation, and in fact, like the phenethylamine, appeared to enhance ADDLs formation.

Initially, the aforementioned testing was performed on nonpurified products because beta-CD derivatives are very difficult to separate. Nevertheless, ESMS and, in some cases, ES LCMS analyses of these reaction mixtures indicated that the per-substituted products were the major component in the reaction mixture, together with small amounts of the partially substituted isomers. In addition, a slower running peak (about 10% of the major per-substituted peak) always existed, whose mass suggested a six substitution pattern with one of the six amines in a tertiary form spanning two positions, probably adjacent, on the primary beta-CD face. A typical HPLC chromatogram for the reaction products is shown in FIG. 3 for the furfurylamine derivative. The aforementioned reaction conditions maximized the per-substituted derivatives, but failed to provide reaction products free from side reaction by-products. Focusing on the two most inhibitory products, i.e., those from the reaction of per-6-iodo-per-6-deoxy-beta-CD with furfurylamine and benzylamine, isolation and testing of purified compounds was initiated.

Attempts to separate the two major products by flash chromatography on silica gel were partially successful, but it was clear that the ADDLs inhibitory activity most likely resided in both the per-substituted and the crosslinked products. Finally, a preparative reversed phase HPLC separation of the per-substituted beta-CD chromatographically produced homogenous products in the case of the per-benzylamino-6-deoxy-beta-CD 5a, its crosslinked derivative 5b, the per-furfurylamino-6-deoxy-beta-CD 4a, and its crosslinked derivative 4b.

These purified derivatives were subjected to full dose response analyses in the ADDLs formation assay (FIG. 5a). As demonstrated, both the furfurylamine derivative 4a and the benzylamine derivative 5a inhibit ADDLs formation with an IC₅₀ of 0.54 and 0.46 μM, respectively. The crosslinked derivatives, in both cases, were found to have similar LD₅₀ inhibitory values, i.e., 4b, 1.0 and 5b, 0.76 μM. In both the crude reactions and as purified derivatives, the furfurylamine beta-CDs were slightly more active than the benzylamine derivatives.

The ADDL inhibitory activity appears to be saturable, as indicated by the sigmoidal concentration response curves, and specific. Thus, corresponding concentrations of the parent beta-CD or the free side chains did not show any detectable inhibitory activity in this assay under the same conditions. In addition, whereas a mixture of the per-6-benzylamino-per-6-deoxy-beta-CD 5a and its crosslinked product 5b were inhibitory for ADDLs formation, as seen in FIG. 5b, the corresponding mixture of per-6-phenyethylaminoper-6-deoxy-beta-CD 6a and its crosslinked product 6b (ratio 80:20 by ESMS) shows dramatically the opposite effect. Thus, at about five times the concentration of the inhibitory effect of its benzylamine counterpart, the phenethylamine derivatives causes a two hundred percent increase in ADDLs formation relative to control.

As an example of the efficacy of per-6-substituted alpha-CDs compared to the above per-6-substituted-beta-CDs, the furfurylamine and benzylamine derivatives were prepared by the method of B. I. Gorin et al., Tet. Letters, 37(27), pages 4647-4650 (1996) and Vizitiu et al., J. Org. Chem., 62(25), pages 8760-8766 (1997) and assayed in the dot-blot assay. The alpha-CDs demonstrated a potency equivalent or superior to the corresponding per-6-substituted-beta-CDs. The utility in the invention of both per-6-substituted-alpha-CDs and per-6-substituted-beta-CDs thereby is demonstrated.

Modifications and variations of the invention as hereinbefore set forth can be made without departing from the spirit and scope thereof, and only such limitations should be imposed as are indicated by the appended claims.

1. A method of treating Alzheimer's disease comprising administering to a mammal in need thereof a therapeutically effective amount of a per-6-substituted-per-6-deoxy-cyclodextrin capable of inhibiting formation or activity of soluble amyloid-beta-derived diffusible ligands.

2. The method of claim 1 wherein the 6-substituted-per-6-deoxy-cyclodextrin comprises a per-6-substituted-per-deoxy-beta-cyclodextrin.

3. The method of claim 1 wherein the 6-substituted-per-β-deoxy-cyclodextrin comprises a per-6-substituted-per-deoxy-alpha-cyclodextrin.

4. The method of claim 1 wherein the per-6-substituted-6-deoxy-cyclodextrin has a structural formula 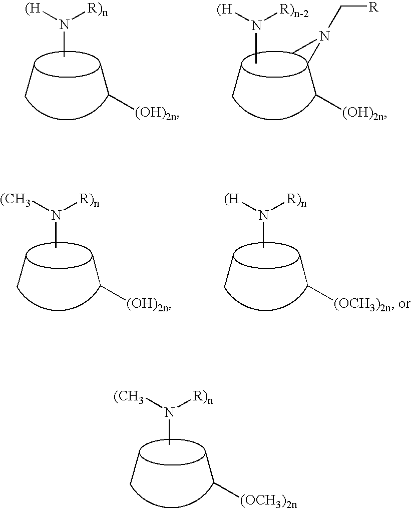
wherein the R group has a structure —CH₂-aryl or —CH₂-heteroaryl, and n is 6 or 7.

5. The method of claim 1 wherein the R group has a structure 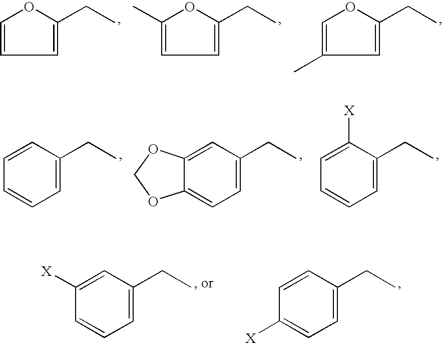
wherein X is selected from the group consisting of Cl, Br, CH₃, C₂H₅, and OCH₃.

6. The method of claim 1 wherein the 6-per-substituted-6-per-deoxy-cyclodextrin lacks an ability to cross the blood-brain barrier.

7. The method of claim 1 further comprising administering a therapeutically effective amount of a second therapeutic agent useful in the treatment of Alzheimer's disease.

8. The method of claim 7 wherein the 6-per-substituted-6-per-deoxy-cyclodextrin and second therapeutic agent are administered simultaneously.

9. The method of claim 7 wherein the 6-per-substituted-6-per-deoxy-cyclodextrin and second therapeutic agent are administered from a single composition.

10. The method of claim 7 wherein the 6-per-substituted-6-per-deoxy-cyclodextrin and second therapeutic agent are administered from separate compositions.

11. The method of claim 7 wherein the 6-per-substituted-6-per-deoxy-cyclodextrin and second therapeutic agent are administered separately.

12. The method of claim 7 wherein the 6-per-substituted-6-per-deoxy-cyclodextrin is administered prior to the second therapeutic agent.

13. The method of claim 7 wherein the 6-per-substituted-6-per-deoxy-cyclodextrin is administered after the second therapeutic agent.

14. The method of claim 1 wherein the mammal is a human.

15. A method of treating a pre-Alzheimer's disease disorder comprising administering to a mammal in need thereof a therapeutically effective amount of a per-6-substituted-per-6-deoxy-cyclodextrin capable of inhibiting formation or activity soluble amyloid-beta-derived diffusible ligands.

16. The method of claim 15 wherein the pre-Alzheimer's disease disorder is mild cognitive impairment.

17. A method of treating a neurodegenerative disease or condition comprising administering to a mammal in need thereof a therapeutically effective amount of a 6-per-substituted-6-deoxy-cyclodextrin capable of inhibiting formation or activity soluble amyloid-beta-derived diffusible ligands.

18. The method of claim 17 wherein the neurodegenerative disease is selected from the group consisting of Parkinson's disease, Huntington's disease, Creutzfeldt-Jacob disease, and a spinocerebellar ataxia.

19. A method of inhibiting formation or activity of an Alzheimer's disease diffusible ligand in a mammal comprising administering a therapeutically effective amount of a 6-per-substituted-6-deoxy-cyclodextrin to the mammal.

20. A compound having a structure 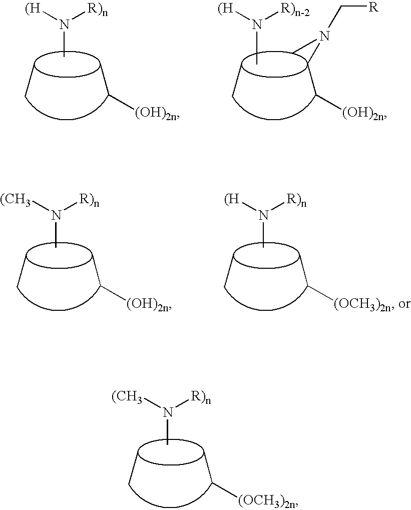
wherein n is 6 or 7, and R is selected from the group consisting of

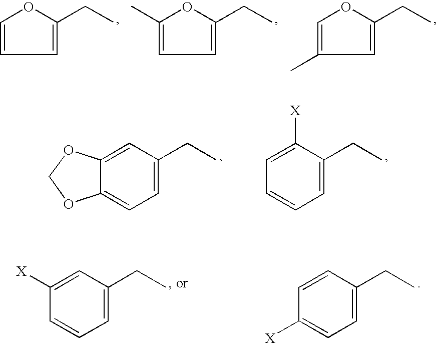

21. A composition comprising a compound of claim 19 and a pharmaceutically effective carrier.

22. A method of reducing neurodegeneration in an individual in need thereof comprising administering a therapeutically effective amount of a per-6-substituted-per-6-deoxy-cyclodextrin capable of inhibiting formation or activity of soluble amyloid-beta-derived diffusible ligands.

23. A method of treating a neurological disorder associated with an aggregation of neurotoxic endogenous peptides comprising administering a therapeutically effective amount of per-6-substituted-per-6-deoxy-cyclodextrin capable of inhibiting formation or activity of soluble amyloid-beta-derived diffusible ligands.
